(19) United States
(12) Patent Application Publication
(10) Pub. No.: US 2025/0221733 A1
(43) Pub. Date: Jul. 10, 2025

(54) CUTTING BLADE WITH FLEXIBLE BALLOON PAD

(71) Applicant: Boston Scientific Scimed, Inc., Maple Grove, MN (US)

(72) Inventor: Gordon John Kocur, Lino Lakes, MN (US)

(73) Assignee: Boston Scientific Scimed, Inc., Maple Grove, MN (US)

(21) Appl. No.: 19/007,871

(22) Filed: Jan. 2, 2025

Related U.S. Application Data
(60) Provisional application No. 63/617,946, filed on Jan. 5, 2024.

(51) Int. Cl. A61B 17/3207; A61B 17/00

(52) U.S. Cl. CPC A61B 17/320725; A61B 2017/00557; A61B 2560/04

(57) ABSTRACT

A medical device useful for cracking or cutting an intravascular lesion may include a blade member including a first blade segment and a second blade segment separated by a blade flex point. The blade member is embedded within a polymeric pad that mounts the blade member to an inflatable balloon. The polymeric pad may include a pad flex point that aligns with the blade flex point in order to allow side bending.

### CROSS REFERENCE TO RELATED APPLICATIONS

The present application claims the benefit of U.S. Provisional Patent Application Ser. No. 63/617,946, filed Jan. 5, 2024, the disclosure of which is incorporated herein by reference.

### TECHNICAL FILED

The disclosure is directed to angioplasty balloon catheters including one or more cutting blades mounted to the balloon. More particularly, the disclosure is directed to cutting blades of a cutting balloon catheter having a more flexible cutting balloon pad.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action, or lack of oxygenation and/or circulation to other regions of the body.

Occluded, stenotic, or narrowed blood vessels, as well as native or synthetic arteriovenous dialysis fistulae, may be treated in a recanalization procedure, such as with an angioplasty balloon catheter advanced over a guidewire to an occlusion so that the balloon is positioned across the occlusion. The balloon is then inflated to enlarge the passageway through the occlusion.

One of the major obstacles in treating coronary artery disease and/or treating blocked blood vessels or fistulae is re-stenosis or re-narrowing of the passageway through the occlusion subsequent to an angioplasty procedure or other recanalization procedure. Evidence has shown that cracking, cutting or scoring the stenosis, for example, with an angioplasty balloon equipped with a blade member, during treatment can reduce incidence of re-stenosis. Additionally, cracking, cutting or scoring the stenosis may reduce trauma at the treatment site and/or may reduce the trauma to adjacent healthy tissue. Blade members may also be beneficial additions to angioplasty procedures when the targeted occlusion is hardened or calcified. It is believed typical angioplasty balloons, alone, may not be able to expand certain of these hardened lesions. Thus, angioplasty balloons equipped with blade members have been developed to attempt to enhance angioplasty treatments.

Blade members may be adapted to cut, crack or score lesions, for example. Blade members are embedded within polymeric pads that are then adhesively secured to an angioplasty balloon. It will be appreciated that an angioplasty balloon may be subjected to bending forces in a variety of directions while a deflated angioplasty balloon is advanced through a patient's vasculature. Blade members may include flex points that allow the blade member to flex when undergoing facial bending as well as side bending. The polymeric pads may be flexible when undergoing facial bending, but may not be as flexible when undergoing side bending. Accordingly, there is an ongoing need for angioplasty balloons with blade members secured via polymeric pads that are more flexible, particularly when being subjected to side bending.

### SUMMARY

The disclosure is directed to several alternative designs, materials and methods of manufacturing medical device structures and assemblies, and the use thereof. An example may be found in a medical device. The medical device includes a catheter shaft having a distal region and an inflatable balloon secured to the distal region. A blade and pad assembly extends longitudinally relative to the inflatable balloon, the blade and pad assembly including a polymeric pad adapted to be adhesively secured to an outer surface of the inflatable balloon and a blade member partially embedded within the polymeric pad. The blade member includes a base portion extending from a first end of the blade member to a second end of the blade member and a blade portion extending outwardly from the base portion. The blade portion including a first blade flex point, a second blade flex point, a first blade segment extending from the first end of the blade member to the first blade flex point, a second blade segment extending from the second end of the blade member to the second blade flex point, and an intermediate blade segment extending between the first blade flex point and the second blade flex point. The polymeric pad includes a first pad flex point aligned with the first blade flex point and a second pad flex point aligned with the second blade flex point.

Alternatively or additionally, the first pad flex point may include a first region of the polymeric pad having a width that is reduced 30 to 70 percent relative to a width of the polymeric pad outside of the first region.

Alternatively or additionally, the second pad flex point may include a second region of the polymeric pad having a width that is reduced 30 to 70 percent relative to a width of the polymeric pad outside of the second region.

Alternatively or additionally, the polymeric pad may taper from a maximum thickness where the blade member is embedded in the polymeric pad to a minimum thickness along either edge of the polymeric pad parallel with the blade member.

Alternatively or additionally, the outer surface of the inflatable balloon, when the inflatable balloon is inflated, may have a balloon radius of curvature in a range of 1 millimeter to 4 millimeters. The polymeric pad has a mounting surface may have a pad radius of curvature in a range of 1 millimeter to 10 millimeters.

Alternatively or additionally, the medical device may further include an adhesive layer disposed between the outer surface of the inflatable balloon and the mounting surface of the polymeric pad.

Alternatively or additionally, the medical device may further include one or more additional blade and pad assemblies extending longitudinally relative to the inflatable balloon, each of the one or more additional blade and pad assemblies including a blade member that includes a base portion extending from a first end of the blade member to a second end of the blade member and a blade portion extending outwardly from the base portion. The blade portion including a first blade flex point, a second blade flex point, a first blade segment extending from the first end of the blade member to the first blade flex point, a second blade segment extending from the second end of the blade member to the second blade flex point, and an intermediate blade segment extending between the first blade flex point and the second blade flex point. The blade and pad assembly includes a polymeric pad adapted to be adhesively secured to the outer surface of the inflatable balloon with the blade member partially embedded within the polymeric pad. The polymeric pad includes a first pad flex point aligned with the first blade flex point and a second pad flex point aligned with the second blade flex point.

Another example may be found in a medical device. The medical device includes an inflatable balloon secured to a distal region of a catheter shaft having a longitudinal axis and a plurality of blade and pad assemblies that are secured to the inflatable balloon and extending longitudinally with respect to the inflatable balloon, each of the plurality of blade assemblies having a longitudinal axis. Each of the blade and pad assemblies include a polymeric pad adapted to be secured to the inflatable balloon and two or more blade segments secured within the polymeric pad, the two or more blade segments separated by one or more blade flex points. The polymeric pad includes one or more pad flex points aligned with the one or more blade flex points.

Alternatively or additionally, each of the one or more pad flex points may be adapted to facilitate side bending of the corresponding polymeric pads.

Alternatively or additionally, each of the one or more pad flex points may include an increased-flexibility portion of the corresponding polymeric pad.

Alternatively or additionally, each of the one or more pad flex points may be adapted to allow adjacent polymeric pads to nest together when the inflatable balloon is deflated and folded.

Alternatively or additionally, each of the one or more pad flex points may include a scalloped portion of the polymeric pad.

Another example may be found in a medical device. The medical device includes an inflatable balloon secured to a distal region of a catheter shaft having a longitudinal axis, the inflatable balloon having an outer surface with, when inflated, a balloon radius of curvature in a range of 1 millimeter to 4 millimeters. The medical device includes a polymeric pad having a mounting surface adapted to be secured to the inflatable balloon, the mounting surface having a pad radius of curvature in a range of 1 millimeter to 10 millimeters. An adhesive layer secures the polymeric pad to the outer surface of the inflatable balloon. A blade member is embedded within the polymeric pad and includes two or more blade segments separated by one or more blade flex points. The polymeric pad includes one or more pad flex points aligned with the one or more blade flex points.

Alternatively or additionally, the adhesive layer may have a uniform thickness.

Alternatively or additionally, the polymeric pad may further include a tapered surface opposite the mounting surface.

Another example may be found in a medical device. The medical device includes a catheter shaft having a distal region and an inflatable balloon that is secured to the distal region. A blade and pad assembly extends longitudinally relative to the inflatable balloon, the blade and pad assembly including a polymeric pad adapted to be adhesively secured to an outer surface of the inflatable balloon and a blade member partially embedded within the polymeric pad. The blade member includes a base portion extending from a first end of the blade member to a second end of the blade member and a blade portion extending outwardly from the base portion. The blade portion includes a first blade flex point, a second blade flex point, a first blade segment extending from the first end of the blade member to the first blade flex point, a second blade segment extending from the second end of the blade member to the second blade flex point, and an intermediate blade segment extending between the first blade flex point and the second blade flex point. The polymeric pad includes a first pad flex point aligned with the first blade flex point and a second pad flex point aligned with the second blade flex point.

Alternatively or additionally, the first blade flex point may include a first gap in the blade portion and the second blade flex point may include a second gap in the blade portion, with the base portion extending without any gap.

Alternatively or additionally, the first pad flex point may include a first region of the polymeric pad having a reduced pad width.

Alternatively or additionally, the first region of the polymeric pad may have a width that is reduced 30 to 70 percent relative to a width of the polymeric pad outside of the first region.

Alternatively or additionally, the second pad flex point may include a second region of the polymeric pad having a reduced pad width.

Alternatively or additionally, the second region of the polymeric pad may have a width that is reduced 30 to 70 percent relative to a width of the polymeric pad outside of the second region.

Alternatively or additionally, the polymeric pad may taper from a maximum thickness where the blade member is embedded in the polymeric pad to a minimum thickness along either edge of the polymeric pad parallel with the blade member.

Alternatively or additionally, the outer surface of the inflatable balloon, when the inflatable balloon is inflated, has a balloon radius of curvature in a range of 1 millimeter to 4 millimeters. The polymeric pad has a mounting surface having a pad radius of curvature in a range of 1 millimeter to 10 millimeters.

Alternatively or additionally, the medical device may further include an adhesive layer disposed between the outer surface of the inflatable balloon and the mounting surface of the polymeric pad.

Alternatively or additionally, the medical device may further include one or more additional blade and pad assemblies extending longitudinally relative to the inflatable balloon, each of the one or more additional blade and pad assemblies including a blade member that includes a base portion extending from a first end of the blade member to a second end of the blade member and a blade portion extending outwardly from the base portion. The blade portion including a first blade flex point, a second blade flex point, a first blade segment extending from the first end of the blade member to the first blade flex point, a second blade segment extending from the second end of the blade member to the second blade flex point, and an intermediate blade segment extending between the first blade flex point and the second blade flex point. The blade and pad assembly includes a polymeric pad adapted to be adhesively secured to the outer surface of the inflatable balloon with the blade member partially embedded within the polymeric pad. The polymeric pad includes a first pad flex point aligned with the first blade flex point and a second pad flex point aligned with the second blade flex point.

Another example may be found in a medical device. The medical device includes an inflatable balloon secured to a distal region of a catheter shaft having a longitudinal axis and a plurality of blade and pad assemblies that are secured to the inflatable balloon and extending longitudinally with respect to the inflatable balloon, each of the plurality of blade assemblies having a longitudinal axis. Each of the blade and pad assemblies include a polymeric pad adapted to be secured to the inflatable balloon and two or more blade segments secured within the polymeric pad, the two or more blade segments separated by one or more blade flex points. The polymeric pad includes one or more pad flex points aligned with the one or more blade flex points.

Alternatively or additionally, each of the one or more pad flex points may be adapted to facilitate side bending of the corresponding polymeric pads.

Alternatively or additionally, each of the one or more pad flex points may include an increased-flexibility portion of the corresponding polymeric pad.

Alternatively or additionally, each of the one or more pad flex points may include a narrowed portion of the corresponding polymeric pad.

Alternatively or additionally, each of the one or more pad flex points may be adapted to allow adjacent polymeric pads to nest together when the inflatable balloon is deflated and folded.

Alternatively or additionally, each of the one or more pad flex points may include a scalloped portion of the polymeric pad.

Another example may be found in a medical device. The medical device includes an inflatable balloon secured to a distal region of a catheter shaft having a longitudinal axis, the inflatable balloon having an outer surface with, when inflated, a balloon radius of curvature in a range of 1 millimeter to 4 millimeters. The medical device includes a polymeric pad having a mounting surface adapted to be secured to the inflatable balloon, the mounting surface having a pad radius of curvature in a range of 1 millimeter to 10 millimeters. An adhesive layer secures the polymeric pad to the outer surface of the inflatable balloon. A blade member is embedded within the polymeric pad and includes two or more blade segments separated by one or more blade flex points. The polymeric pad includes one or more pad flex points aligned with the one or more blade flex points.

Alternatively or additionally, the adhesive layer may have a uniform thickness.

Alternatively or additionally, the polymeric pad may further include a tapered surface opposite the mounting surface.

Alternatively or additionally, each of the one or more pad flex points may include a region of the polymeric pad having a width that is reduced 30 to 70 percent relative to a width of the polymeric pad away from the pad flex point.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partially cross-sectioned schematic of an example medical device;

FIG. 2 is a schematic view of a blade member usable in the example medical device of FIG. 1;

FIG. 3 is a schematic top view of an example blade and pad assembly;

FIG. 4 is a schematic side view of the example blade and pad assembly of FIG. 3;

FIG. 5 is a schematic top view of an example blade and pad assembly shown in a linear configuration, with pad flex features aligned with the blade flex features;

FIG. 6 is a schematic top view of the example blade and pad assembly of FIG. 5, shown in an angled configuration bending at a flex feature;

FIG. 7 is a cross-sectional view taken along the line 7-7 of FIG. 6;

FIG. 8 is a cross-sectional view taken along the line 8-8 of FIG. 6;

FIG. 9 is a schematic view of an example blade and tapered thickness pad assembly;

FIG. 10 is a schematic view showing how the example blade and pad assembly may overlap with another example blade and pad assembly when an inflatable balloon bearing the example blade assemblies is deflated and folded;

FIG. 11 is a schematic view of an example blade and pad assembly;

FIG. 12 is a schematic view showing several example blade assemblies of FIG. 11 nested together when an inflatable balloon bearing the example blade assemblies is deflated and folded;

FIG. 13 is a schematic view of an example blade and curved base pad assembly mounted to an inflatable balloon; and

FIG. 14 is a schematic view of an example blade and curved base pad assembly mounted to an inflatable balloon.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

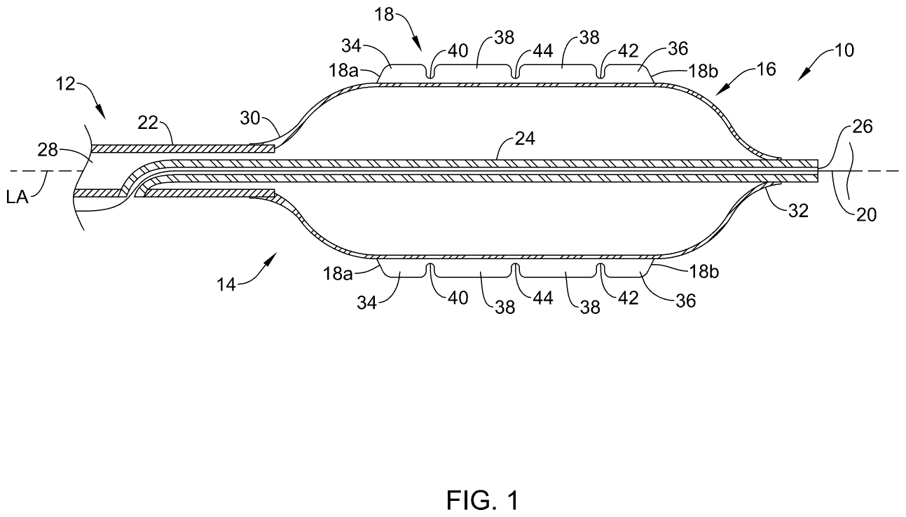

FIG. 1 is a partial cross-sectional side view of an example medical device 10. In some instances, the example medical device 10 may be utilized within a patient's vasculature in order to treat a stenosis within the vasculature. In some instances, the medical device 10 may be used to crack or cut a resistant lesion. The medical device 10 may define a longitudinal axis “LA”, as shown. The medical device 10 includes a catheter shaft 12 having a distal region 14. An inflatable balloon 16 may be secured to the distal region 14 of the catheter shaft 12. One or more blade members 18 may be mounted on or otherwise secured relative to the inflatable balloon 16. In some instances, the medical device 10 may be advanced over a guidewire 20, through the vasculature, to a target area. Once positioned at the target location in the vasculature, the inflatable balloon 16 can be inflated to exert a radially outward force on a lesion, as the blade members 18 engage the lesion. Thus, the blade members 18 may crack, cut or score the lesion to facilitate enlarging the lumen proximate the lesion. The target area may be within any suitable peripheral or cardiac vessel lumen location.

The blade members 18 may vary in number, position, and arrangement about the inflatable balloon 16. For example, the medical device 10 may include one, two, three, four, five, six, or more blade members 18 that are disposed at any position along the inflatable balloon 16 and in a regular, irregular, or any other suitable pattern. For example, in some instances, the inflatable balloon 16 may include a plurality of blade members 18 longitudinally arranged symmetrically around a circumference of the inflatable balloon 16.

The blade members 18 may be made from any suitable material such as a metal, metal alloy, polymer, metal-polymer composite, and the like, or any other suitable material. For example, the blade members 18 may be made from stainless steel, titanium, nickel-titanium alloys, tantalum, iron-cobalt-nickel alloys, or other metallic materials in some instances. The blade members 18 may have a triangular cross-sectional shape, or a square cross-sectional shape, or a rectangular cross-sectional shape, a circular cross-sectional shape or any other desired configuration.

The blade members 18 may be mounted to the inflatable balloon 16 using a variety of techniques. In some cases, each of the blade members 18 may include a base portion that includes cutouts for flexibility, where the base portion may be at least partially embedded in a polymeric member (not shown in FIG. 1) that may itself be secured to an outer surface of the inflatable balloon 16. Additional details regarding how the blade member 18 may be secured to the outer surface of the inflatable balloon 16, including the aforementioned polymeric member, are described for example in U.S. Pat. Nos. 9,226,768; 10,046,146; 10,058,349; and 10,729,893, which are incorporated by reference in their entireties.

The inflatable balloon 16 may be made from angioplasty balloon materials including polymers such as polyethylene terephthalate (PET), polytetrafluoroethylene (PTFE), ethylene tetrafluoroethylene (ETFE), polybutylene terephthalate (PBT), polyurethane, polyvinylchloride (PVC), polyether-ester, polyester, polyamide, elastomeric polyamides, polyether block amide (PEBA), as well as other suitable materials, or mixtures, combinations, copolymers thereof, polymer/metal composites, and the like. In some instances, the inflatable balloon 16 may include a single layer of material, whereas in other instances the inflatable balloon 16 may be of a multi-layer construction, including a plurality of layers of materials. For instance, the inflatable balloon 16 may be formed as a co-extrusion or tri-layer extrusion in some instances.

The inflatable balloon 16 may be configured so that the inflatable balloon 16 includes one or more “wings” or wing-shaped regions when the inflatable balloon 16 is deflated. In some instances, the wings may be configured so that the blade members 18 can be positioned at the inward-most positions of the deflated inflatable balloon 16, with the wings of the balloon folds positioned between adjacent blade members 18. This arrangement may minimize the crossing profile of the folded inflatable balloon 16 and reduce the exposure of the blade members 18 to the blood vessel during delivery of the inflatable balloon 16 to the lesion.

The catheter shaft 12 may be a catheter shaft, similar to typical catheter shafts. For example, the catheter shaft 12 may include an outer tubular member 22 and an inner tubular member 24 extending through at least a portion of the outer tubular member 22. The outer tubular member 22 and the inner tubular member 24 may each be manufactured from a number of different materials. For example, the outer tubular member 22 and the inner tubular member 24 may be made of metals, metal alloys, polymers, metal-polymer composites or any other suitable materials.

The outer tubular member 22 and the inner tubular member 24 may be arranged in any appropriate way. For example, in some embodiments the inner tubular member 24 can be disposed coaxially within the outer tubular member 22. According to these embodiments, the inner tubular member 24 and the outer tubular member 22 may or may not be secured to one another along the general longitudinal axis of the catheter shaft 12. Alternatively, the inner tubular member 24 may follow the inner wall or otherwise be disposed adjacent the inner wall of the outer tubular member 22. In other embodiments, the outer tubular member 22 and the inner tubular member 24 may be arranged in another desired fashion.

The inner tubular member 24 may include an inner lumen 26. In at least some instances, the inner lumen 26 is a guidewire lumen for receiving the guidewire 20 therethrough. Accordingly, the medical device 10 can be advanced over the guidewire 20 to the desired location. The guidewire lumen 26 may extend along essentially the entire length of the catheter shaft 12 such that the medical device 10 resembles a traditional “over-the-wire” catheter. Alternatively, and as shown, the guidewire lumen 26 may extend along only a portion of the catheter shaft 12 such that the medical device 10 resembles a “single-operator-exchange” or “rapid-exchange” catheter.

The catheter shaft 12 may also include an inflation lumen 28 that may be used, for example, to transport inflation media to and from the inflatable balloon 16 to selectively inflate and/or deflate the inflatable balloon 16. The location and position of the inflation lumen 28 may vary, depending on the configuration of the outer tubular member 22 and the inner tubular member 24. For example, when the outer tubular member 22 surrounds the inner tubular member 24, the inflation lumen 28 may be defined within the space between the outer tubular member 22 and the inner tubular member 24. In instances in which the outer tubular member 22 is disposed alongside the inner tubular member 24, then the inflation lumen 28 may be the lumen of the outer tubular member 22.

The inflatable balloon 16 may be coupled to the catheter shaft 12 in any of a number of suitable ways. For example, the inflatable balloon 16 may be adhesively or thermally bonded to the catheter shaft 12. In some embodiments, a proximal waist 30 of the inflatable balloon 16 may be bonded to the catheter shaft 12, for example, bonded to a distal end of the outer tubular member 22, and a distal waist 32 of the inflatable balloon 16 may be bonded to the catheter shaft 12, for example, bonded to a distal end of the inner tubular member 24. The exact bonding positions, however, may vary.

Each of the blade members 18 may be considered as including a first terminal blade segment 34 at a first end of the blade member 18 and a second terminal blade segment 36 at an opposite, second end of the blade member 18. As illustrated, the first terminal blade segment 34 corresponds to a proximal-most blade segment while the second terminal blade segment 36 corresponds to a distal-most blade segment. In some instances, the first terminal blade segment 34 may correspond to a distal-most blade segment and the second terminal blade segment 36 may correspond to a proximal-most blade segment. The first terminal blade segment 34 extends from a first end 18a of the blade member 18 to a first blade flex point 40 that is formed in the blade member 18. The second terminal blade segment 36 extends from a second end 18b of the blade member 18 to a second blade flex point 42 that is formed in the blade member 18.

In some instances, the first blade flex point 40 and the second blade flex point 42, the relative position of which may be thought of as defining a first terminal blade segment length and a second terminal blade segment length, respectively, are positioned such that the first blade flex point 40 is within two millimeters of a first end of a lesion when the second blade flex point 42 is within two millimeters of a second end of the lesion. In some instances, the first terminal blade segment 34 may have a length of less than three millimeters. In some instances, the first terminal blade segment 34 may have a length of less than two millimeters. In some instances, the second terminal blade segment 36 may have a length of less than three millimeters. In some instances, the second terminal blade segment 36 may have a length of less than two millimeters. These are just examples. In some instances, the first terminal blade segment 34 and/or the second terminal blade segment 36 may have greater lengths.

Each of the blade members 18 may include one or more intermediate blade segments 38. As shown, each blade member 18 includes two intermediate blade segments 38, separated by a third blade flex point 44. In some instances, the intermediate blade segments 38 may be the same length as each of the first terminal blade segment 34 and the second terminal blade segment 36. In some instances, the intermediate blade segments 38 may be shorter in length than the first terminal blade segment 34 and the second terminal blade segment 36. In some instances, as shown, the intermediate blade segments 38 may be longer than either the first terminal blade segment 34 or the second terminal blade segment 36. In some instances, each blade member 18 may only include one intermediate blade segment 38, and thus would not include the third blade flex point 44. In some instances, each blade member 18 may include three or more intermediate blade segments 38, and thus may include a fourth blade flex point or even a fifth blade flex point. It will be appreciated that the number of intermediate blade segments 38 may be determined at least in part upon a desired overall length for each blade member 18. In some instances, the inflatable balloon 16 may include two or more blade members 18 each having the same overall length. In some instances, the inflatable balloon 16 may include two or more blade members 18 each having a different overall length.

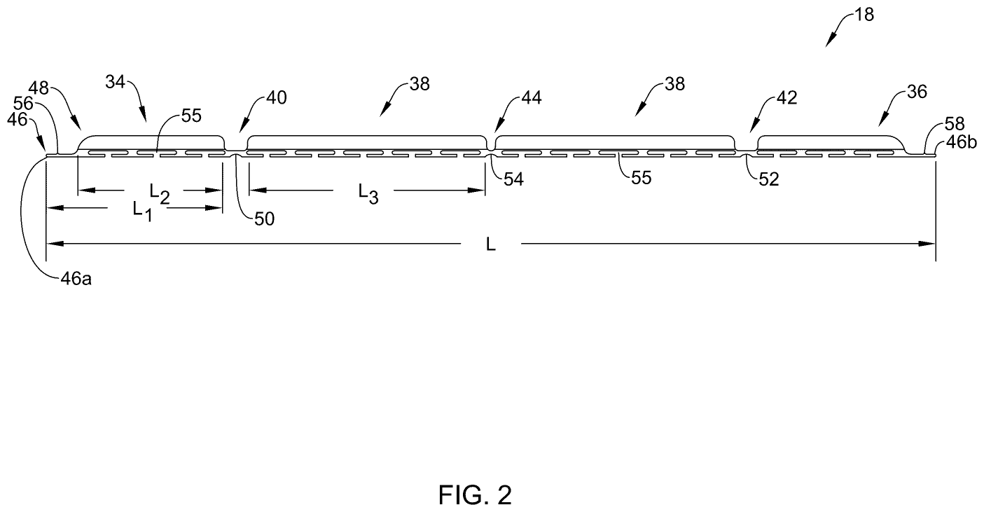

FIG. 2 is a side view of one of the blade members 18. The blade member 18 shown in FIG. 2 may be considered as being the same as the blade member 18 shown in FIG. 1. The blade member 18 includes a base portion 46 that extends from a first end 46a of the blade member 18 to a second end 46b of the blade member 18. The blade member 18 includes a blade portion 48 that extends outwardly from the base portion 46. The blade portion 48 includes the first terminal blade segment 34, the second terminal blade segment 36 and the intermediate blade segments 38, separated by the first blade flex point 40, the second blade flex point 42 and the third blade flex point 44, respectively.

In some instances, as shown, the first blade flex point 40 and the second blade flex point 42 may be wider than the third blade flex point 44 (or any additional intervening blade flex points). The first blade flex point 40 and the second blade flex point 42 may each be ten percent wider or more, or twenty percent wider or more, or thirty percent wider or more, or forty percent wider or more, or fifty percent wider or more, or sixty percent wider or more, or seventy percent wider or more, or eighty percent wider or more, or 90 percent wider or more, or 100 percent wider or more than the third blade flex point 44. In some instances, having the first blade flex point 40 and the second blade flex point 42 being wider than the third blade flex point 44 may allow the first terminal blade segment 34 and the second terminal blade segment 36, respectively, to flex to a relatively greater angle relative to adjacent intermediate blade segments 38 without the first terminal blade segment 34 and/or the second terminal blade segment 36 hitting or otherwise being interfered with by the adjacent intermediate blade segments 38. In some instances, the first blade flex point 40 and/or the second blade flex point 42 may be adapted to permit the first terminal blade segment 34 and/or the second terminal blade segment 36 to flex to a position in which the first terminal blade segment 34 and/or the second terminal blade segment 36 forms an angle of up to 90 degrees with respect to the intermediate blade segments 38.

In some instances, the first blade flex point 40 may be considered as being a first gap in the blade portion 48 (e.g., a gap between adjacent portions of the cutting edge of the blade portion 48), the second blade flex point 42 may be considered as being a second gap in the blade portion 48 (e.g., a gap between adjacent portions of the cutting edge of the blade portion 48) and the third blade flex point 44 may be considered as being a third gap in the blade portion 48 (e.g., a gap between adjacent portions of the cutting edge of the blade portion 48). In some cases, as shown, the gaps extend through the blade portion 48 but do not extend through the base portion 46, meaning that the blade member 18 is a single, unitary structure, formed from a single piece of material.

In some instances, the base portion 46 may include a thinned region that corresponds to the position of the blade flex points 40, 42 and 44. As shown, the base portion 46 includes a first thinned region 50 that aligns with the first blade flex point 40, a second thinned region 52 that aligns with the second blade flex point 42 and a third thinned region 54 that aligns with the third blade flex point 44. It will be appreciated that the thinned regions 50, 52 and 54 cooperate with the gaps corresponding to the blade flex points 40, 42 and 44 to further improve the flexibility of the blade member 18. The thinned regions 50, 52, 54 may also be considered living hinges between adjacent blade segments 34, 36, 38, allowing the blade member 18 to flex or bend at the living hinges or thinned regions 50, 52, 54. Flexibility of the blade member 18 helps with being able to navigate small or heavily curved portions of the vasculature as well as with allowing the terminal blade segments 34 and 36 to bend or flex further with respect to the intermediate blade segments 38.

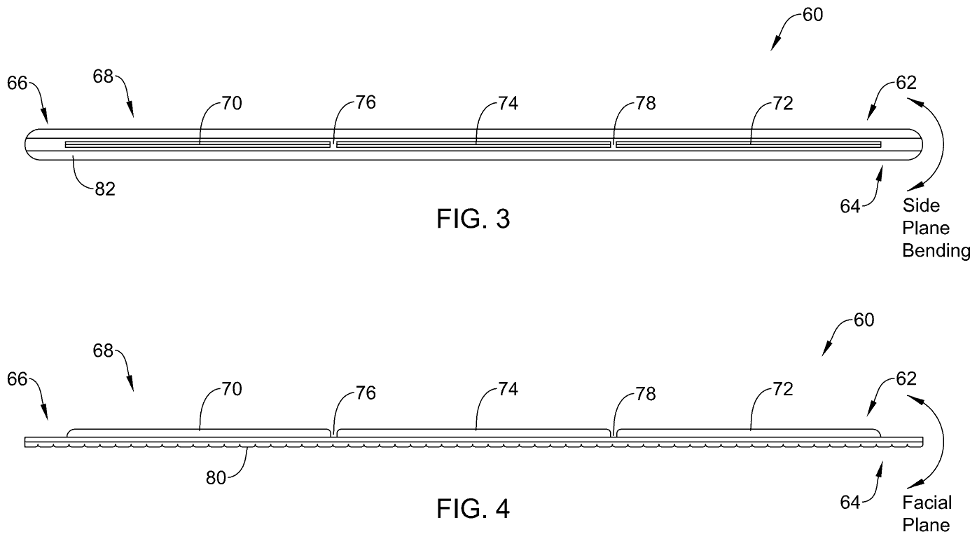

In some instances, the base portion 46 may include a number of T-shaped slots or cutouts 55 that help to further improve the flexibility of the blade member 18. The T-shaped cutouts 55 also help to anchor the base portion 46 within a polymeric member or pad (as shown for example in FIGS. 3 through 14) that is itself secured to the inflatable balloon 16 in order to anchor the blade member 18 relative to the inflatable balloon 16. In some instances, the material of the polymeric member or pad flows into the T-shaped cutouts 55 to further anchor the blade member 18 relative to the inflatable balloon 16. Further details regarding the T-shaped cutouts 55 and their functionality are described in U.S. Pat. Nos. 9,226,768; 10,046,146; 10,058,349; and 10,729,893, which were previously incorporated by reference. In some cases, there will be at least one or more T-shaped cutouts 55 in each of the blade segments, including terminal blade segments.

In the example shown in FIG. 2, the blade member 18 has an overall length “L”. As shown, the blade member 18 has an overall length L that is equal to about 10 millimeters. In some instances, the blade member 18 may instead have an overall length L of about 6 millimeters. In some instances, the blade member 18 may have an overall length L of about 14 or 15 millimeters. It will be appreciated that these examples are merely illustrative, as the blade members may have any desired length.

It can be seen that the base portion 46 extends beyond the blade portion 48 at the first end 46a and at the second end 46b, with the base portion 46 including a first tail piece 56 at the first end 46a and a second tail piece 58 at the second end 46b. In some instances, the additional length provided by the first tail piece 56 and the second tail piece 58 may be useful in securing the base portion 46 (and hence the blade member 18) relative to the inflatable balloon 16. In some instances, the first terminal blade segment 34 may be considered as having a length “L₁” (including the tail piece 56). In some instances, the first terminal blade segment 34 may be considered as having a length “L2” that excludes the tail piece 56. While not labeled, in some instances the second terminal blade segment 36 may be considered as having similar dimensions.

In some instances, L₁ and L₂ may each be 3 millimeters or less. In some instances, L₁ and L₂ may each be 2 millimeters or less. In a particular example, when L is 15 millimeters, L₁ may be equal to about 2 millimeters, such as 2.07 millimeters, and L₂ may be equal to about 1.75 millimeters, such as 1.74 millimeters. Each of the intermediate blade segments 38 may be considered as having a length “L₃” that is defined as the distance between the blade flex points located at either end of the intermediate blade segment 38, such as the first blade flex point 40 and the third blade flex point 44, or the second blade flex point 42 and the third blade flex point 44. L₃ may be adjusted to provide a desired overall length for the blade member 18. As an example, when L is equal to about 10 millimeters, each of the intermediate blade segments 38 may have a length L₃ that is equal to about 2.75 millimeters, such as 2.77 millimeters. Other relative lengths are also contemplated.

FIG. 3 is a schematic top view of an example blade and pad assembly 60 and FIG. 4 is a schematic side view of the example blade and pad assembly 60. The blade and pad assembly 60 may incorporate the blade member 18 shown in FIG. 1, for example, and provides an example of the polymeric pad not shown in FIGS. 1 and 2. The blade and pad assembly 60 includes a blade member 62, such as the blade member 18 shown in FIG. 1, that is secured within a polymeric pad 64. The blade member 62 may be considered as including a base portion 66 and a blade portion 68. The base portion 66 may be similar to the base portion 46 discussed with respect to FIG. 2, and may for example include the same T-shaped slots or cutouts 55 in order to increase the flexibility of the blade member 62 as well as to improve contact with the polymeric pad 64. The base portion 66 is at least partially embedded within the polymeric pad 64, and thus the T-shaped slots or cutouts 55 are not visible in this view.

The blade portion 68 includes a first terminal blade segment 70, a second terminal blade segment 72 and an intermediate blade segment 74 that is disposed between the first terminal blade segment 70 and the second terminal blade segment 72. In this example, the first terminal blade segment 70, the second terminal blade segment 72 and the intermediate blade segment 74 are each the same length. In some instances, one or more of the first terminal blade segment 70, the second terminal blade segment 72 and the intermediate blade segment 74 may be different lengths. In some instances, there may be more than one intermediate blade segment 74, depending on the overall length of the blade member 62. In some instances, the blade portion 68 may include the first terminal blade segment 70 and the second terminal blade segment 72, but may not include the intermediate blade segment 74.

The blade portion 68 includes a first blade flex point 76 that is disposed between the first terminal blade segment 70 and the intermediate blade segment 74 and a second blade flex point 78 that is disposed between the intermediate blade segment 74 and the second terminal blade segment 72. In some instances, the first blade flex point 76 may be considered as being a first gap in the blade portion 68 (e.g., a gap between adjacent portions of the cutting edge of the blade portion 68) and the second blade flex point 78 may be considered as being a second gap in the blade portion 68 (e.g., a gap between adjacent portions of the cutting edge of the blade portion 68). In some cases, the gaps extend through the blade portion 68 but do not extend through the base portion 66, meaning that the blade member 62 is a single, unitary structure, formed from a single piece of material.

As noted, the polymeric pad 64 helps to secure the blade member 62 relative to an outer surface of the inflatable balloon 16. In some instances, the polymeric pad 64 may be adhesively secured to the outer surface of the inflatable balloon 16. The polymeric pad 64 may be considered as having a mounting surface 80 (seen in FIG. 4) as well as an outward-facing surface 82 (seen in FIG. 3). In some instances, the mounting surface 80 may be a smooth surface. In some instances, as shown, the mounting surface 80 may be a textured surface. In some instances, having a textured surface provides additional surface for an adhesive to spread and adhere between the polymeric pad 64 and the balloon surface. In some instances, having a textured or channeled bonding surface may ensure a specified distance between the blade embedded in the pad and the balloon surface to protect the balloon from blade contact and damage. The polymeric pad 64 may be formed of any suitable polymeric material, particularly those commonly used in medical devices. In some instances, the polymeric pad 64 may be formed by injection molding polymers around the blade member 62 to the desired shape. The shape of the polymeric pad 64 may be formed using molds with curable polymers. In some instances, the shape of the polymeric pad 64 may be formed by a stamping or cutting process. A wide range of polymers may be used that provide particular processing and mechanical properties useful in enabling blade attachment to the polymeric pad 64.

In some instances, the polymeric pad 64 may be a unitary polymeric structure, meaning that the polymeric pad 64 extends from a distal end to a proximal end as a single element. In some instances, the polymeric pad 64 may be considered as having a length in a direction parallel with the blade member 62, a width in a direction orthogonal to the blade member 62, and a thickness in a direction orthogonal to the width. The thickness may refer to a distance between the mounting surface 80 and the outward-facing surface 82. In some instances, the polymeric pad 64 has an overall or average width that is sufficient to prevent or reduce the possibility of the blade and pad assembly 60 tipping with respect to an outer surface of the inflatable balloon 16. In some instances, the thickness of the polymeric pad 64, particularly along a midline of the polymeric pad 64, may be limited by the dimensions of the base portion 66 of the blade member 62. The polymeric pad 64 may be thick enough to accommodate the base portion 66, while minimizing the deflated and folded dimensions of the inflatable balloon 16.

Because the thickness is relatively thin, the polymeric pad 64 may be considered as being flexible when the blade and pad assembly 60 is undergoing facial plane bending. Facial plane bending means, for example, that the second terminal blade segment 72 is flexing radially toward or away from the central longitudinal axis of the inflatable balloon 16 that the blade and pad assembly 60 is mounted on (i.e., moving in or out of the page relative to the plane of the paper in FIG. 3 or moving left or right within the plane of the paper in FIG. 4). However, the polymeric pad 64 as shown may not be as flexible when the blade and pad assembly 60 is undergoing side plane bending. Side plane bending means, for example, that the second terminal blade segment 72 is flexing laterally relative to the longitudinal axis of the blade and pad assembly 60 (i.e., moving left or right within the plane of the paper in FIG. 3, or moving in or out of the page relative to the plane of the paper in FIG. 4). The first blade flex point 76 and the second blade flex point 78 are adapted to allow the blade member 62 to be flexible in multiple directions. As the inflatable balloon 16 including one or more blade and pad assemblies 60 is advanced through a patient's vasculature, the blade and pad assemblies 60 will be forced to bend in multiple planes in order to fit through the patient's vasculature. For a particular bend in the vasculature, one of the blade and pad assemblies 60 may undergo facial plane bending, another of the blade and pad assemblies 60 may undergo side plane bending and another of the blade and pad assemblies 60 may undergo a combination of facial plane bending and side plane bending. In some instances, each of the blade and pad assemblies 60 may undergo a combination of facial plane bending and side plane bending.

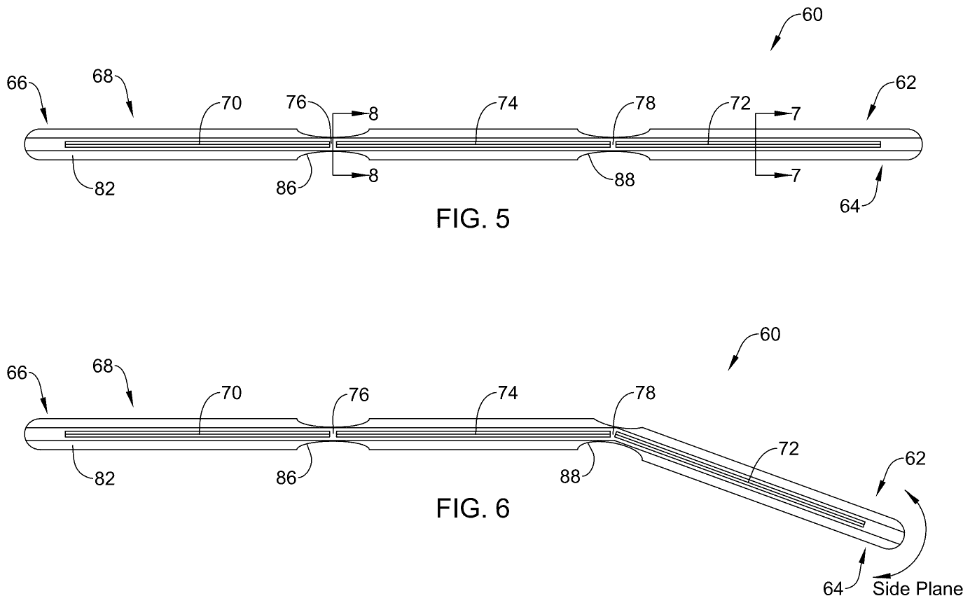

In some instances, the polymeric pad 64 may be adapted to more easily bend. In some instances, the polymeric pad 64 may include pad flex points that are adapted to facilitate side plane bending of the polymeric pad 64, and hence side plane bending of the blade member 62 and blade and pad assembly 60. FIG. 5 is a top schematic view of the blade and pad assembly 60 showing the addition of several pad flex points. In some instances, as shown, the polymeric pad 64 may include a first pad flex point 86 that is aligned with (i.e., longitudinally aligned with) the first blade flex point 76 and a second pad flex point 88 that is aligned with (i.e., longitudinally aligned with) the second blade flex point 78. The first pad flex point 86 may be adapted to facilitate side plane bending. The first pad flex point 86 may be adapted to provide increased flexibility to the polymeric pad 64. In some instances, the first pad flex point 86 may correspond to a tapered or notched out region of the polymeric pad 64 that has a reduced width (in a direction perpendicular to the longitudinal axis of the blade and pad assembly 60) relative to a width of the polymeric pad 64 located on either side of the reduced width portion. The second pad flex point 88 may be adapted to facilitate side plane bending. The second pad flex point 88 may be adapted to provide increased flexibility to the polymeric pad 64. In some instances, the second pad flex point 88 may correspond to a tapered or notched out region of the polymeric pad 64 that has a reduced width (in a direction perpendicular to the longitudinal axis of the blade and pad assembly 60) relative to a width of the polymeric pad 64 located on either side of the reduced width portion.

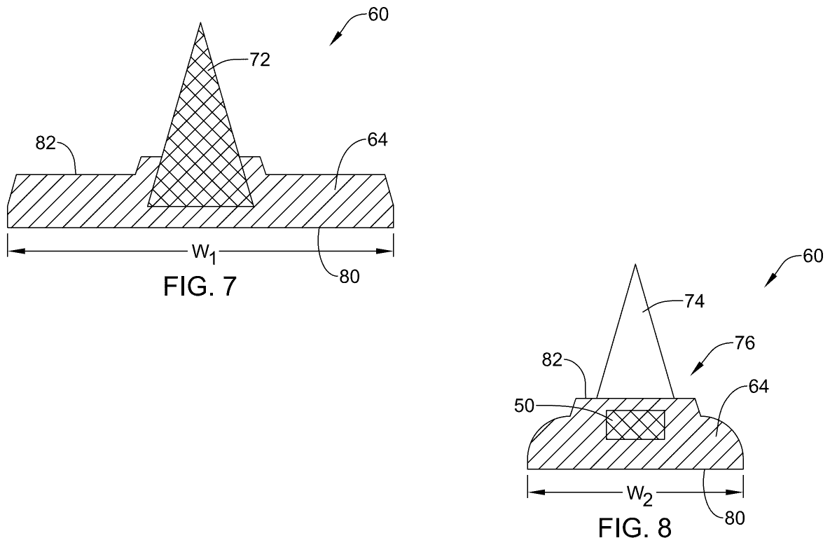

FIG. 6 is a top schematic view of the blade and pad assembly 60, showing the blade and pad assembly 60 undergoing side plane bending at the second blade flex point 78 and the second pad flex point 88 that is aligned with the second blade flex point 78. FIG. 7 is a cross-sectional view taken along the line 7-7 of FIG. 5 in a plane perpendicular to the longitudinal axis of the blade member 18/62, and shows a cross-section through the second terminal blade segment 72 and the polymeric pad 64. FIG. 8 is a cross-sectional view taken along the line 8-8 of FIG. 5 in a plane perpendicular to the longitudinal axis of the blade member 18/62, and shows a cross-section through the first blade flex point 76 and thus through the living hinge or thinned region 50 and the polymeric pad 64, looking at the intermediate blade segment 74. In other words, the cross-section shown in FIG. 8 is taken between adjacent blade segments 74. As shown in FIG. 8, the thinned region 50 of the blade member 18 may be fully encapsulated by the polymeric pad 64 (i.e., fully surrounded by the polymeric pad 64), in some instances. In other embodiments, the thinned region 50 of the blade member 18/62 may be partially encapsulated by the polymeric pad 64 or may be positioned above the upper surface of the polymer pad 64 if desired. It is noted that FIG. 7 is a cross-sectional view representative of a cross-section taken through any one of the blade segments, and FIG. 8 is a cross-sectional view representative of a cross-section taken through any one of the blade flex points 76 (i.e., through any one of the thinned regions).

As shown in FIG. 7, the polymeric pad 64 has a width “W₁” outside of either the first pad flex point 86 or the second pad flex point 88. As used herein, “outside of a pad flex point” is intended to mean along a longitudinal portion of the length of the blade and pad assembly 60 that extends beyond or lacks a pad flex point. As shown in FIG. 8, the polymeric pad 64 has a width “W₂” within the first pad flex point 86. As used herein, “within of a pad flex point” is intended to mean along a longitudinal portion of the length of the blade and pad assembly 60 that includes a pad flex point. In some instances, the width “W₁” may range from 0.3 millimeters to 2 millimeters and the width “W₂” may range from 0.1 millimeters to 0.6 millimeters. In some instances, the width “W₂” may represent a reduction in width, relative to the width “W₁”, of 30 percent to 70 percent, for example. In some instances, the reduction represented by the width “W₂” may be limited by the dimensions of the base portion 66 of the blade member 62 embedded within the polymeric pad 64. In some instances, the first pad flex point 86 and the second pad flex point 88 may each have an overall length, measured between a point before each pad flex point where the width of the polymeric pad 64 returns to its full width and a point after each pad flex point where the width of the polymeric pad 64 returns to its full width. The overall length of the first pad flex point 86 and the second pad flex point 88 may range from 5 percent to 100 percent of the blade segment length.

Referring to FIG. 5 and FIG. 6, the blade and pad assembly 60 shows a first blade flex point 76 and a first pad flex point 86 that are longitudinally aligned with each other as well as a second blade flex point 78 and a second pad flex point 88 that are longitudinally aligned with each other. In some instances, depending on the overall length of the blade member 62, the blade and pad assembly 60 may include a third blade flex point and a third pad flex point that is longitudinally aligned with the third blade flex point. The blade and pad assembly 60 may include a fourth blade flex point and a fourth pad flex point that is longitudinally aligned with the fourth blade flex point. In some instances, the blade and pad assembly 60 may include only a single blade flex point and a single pad flex point that is longitudinally aligned with the single blade flex point.

In some instances, each of the pad flex points, such as the first pad flex point 86 and the second pad flex point 88 may be molded into the polymeric pad 64 when the polymeric pad 64 is molded. In some instances, each of the pad flex points, such as the first pad flex point 86 and the second pad flex point 88 may be cut or stamped into the polymeric pad 64 after the polymeric pad 64 is molded. The polymeric pad 64 may be molded with the base portion 66 of the blade member 62 disposed within the mold such that the base portion 66 becomes embedded within the polymeric pad 64.

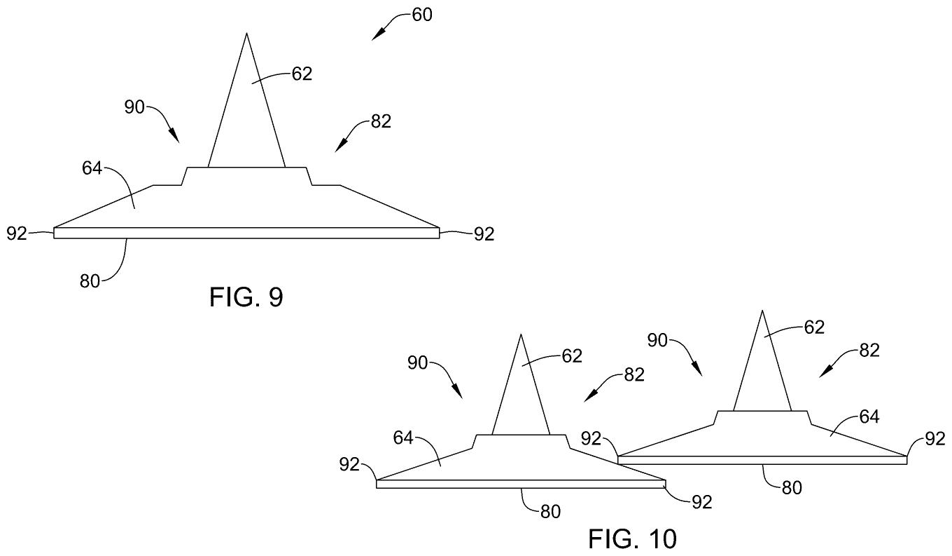

As noted, the inflatable balloon 16 may be deflated and folded over on itself prior to when the inflatable balloon 16 is inflated, including when the medical device 10 including the inflatable balloon 16 is advanced through the patient's vasculature. In some instances, depending on the dimensions of the inflatable balloon 16 and the blade and pad assemblies 60, the polymeric pads 64 may overlap each other when the inflatable balloon 16 is deflated and folded. FIG. 9 is a schematic view of the blade and pad assembly 60, modified with a tapered thickness to allow the polymeric pads 64 to fit more closely together when the inflatable balloon 16 is deflated and folded. In FIG. 9, the outward-facing surface 82 can be seen as tapering from a maximum thickness near a point 90, where the blade member 62 is embedded in the polymeric pad 64, to a minimum thickness along either edge 92 of the polymeric pad 64. The edges 92 may extend longitudinally along opposite lateral extents of the polymeric pad 64. The edges 92 may be considered as being parallel with the blade member 62. Having a minimum thickness along the edges 92 means that a combined thickness of two polymeric pads 64 when the polymeric pads 64 overlap is minimized, as shown for example in FIG. 10. If the profile of the polymeric pad 64 as shown for example in FIG. 7 is superimposed on FIG. 10, it can be appreciated that tapering the polymeric pads 64 saves considerable space, allowing for the inflatable balloon 16 to be more tightly folded over on itself when deflated. A pad with tapered edges may be more flexible in facial plane bending and side plane bending, thereby improving folded device deliverability. Moreover, in some cases, having a tapered outward-facing surface 82 such as that shown in FIG. 9 may be implemented with the polymeric pad 64 shown in earlier drawings.

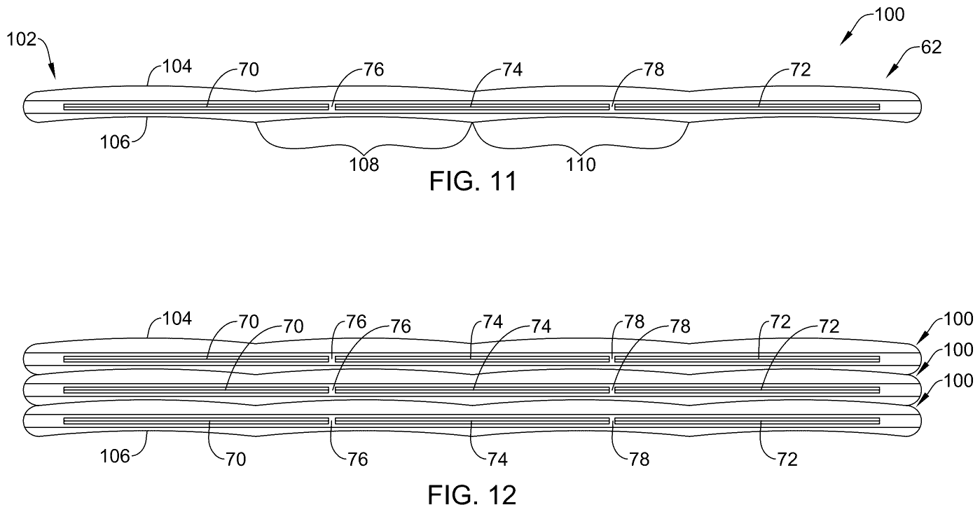

The pad flex points 86 and 88 shown in FIGS. 5 and 6 are one example of a profile for the pad flex points. In some instances, the pad flex points may be part of a scalloped profile for the polymeric pad 64 that allows the polymeric pads 64 from each of several blade and pad assemblies 60 to nest together side by side when the inflatable balloon 16 is deflated and folded over on itself. FIG. 11 is a schematic top view of a blade and pad assembly 100 that includes the blade member 62 mounted in a polymeric pad 102. The polymeric pad 102 may be seen as having a first non-linear edge, such as a first scalloped edge 104, and a second non-linear edge, such as a second scalloped edge 106. The first scalloped edge 104 may include a plurality of arcuate edge segments, such as convexly curved arcuate edge segments. The second scalloped edge 106 may include a plurality of arcuate edge segments, such as concavely curved arcuate edge segments. The first scalloped edge 104 is generally farther from the blade member 62 and the second scalloped edge 106 is generally closer to the blade member 62. The first scalloped edge 104 is farthest from the blade member 62 where the second scalloped edge 106 is closest to the blade member 62, and vice versa. The second scalloped edge 106 defines a first pad flex point 108 that is aligned with or centered on the first blade flex point 76 and a second pad flex point 110 that is aligned with or centered on the second blade flex point 78. FIG. 12 shows a number of blade assemblies 100 nested side by side together, as they would when the inflatable balloon 16 is deflated and folded over on itself. In some instances, the profiles shown for the polymeric pad 102 may favor nesting and a minimized folded configuration over facilitating pad flexibility, for example. For instance, the first scalloped edge 104 of the polymeric pad 102 of one blade and pad assembly 100 may mate with the second scalloped edge 106 of a polymeric pad 102 of an adjacent blade and pad assembly 100 when the inflatable balloon is in a deflated state.

In other instances, the first non-linear edge of a first one of the polymeric pads 102 may mate with the second non-linear edge of a second one of the polymeric pads 102 such that the first and second polymeric pads 102 nest against one another in a different fashion.

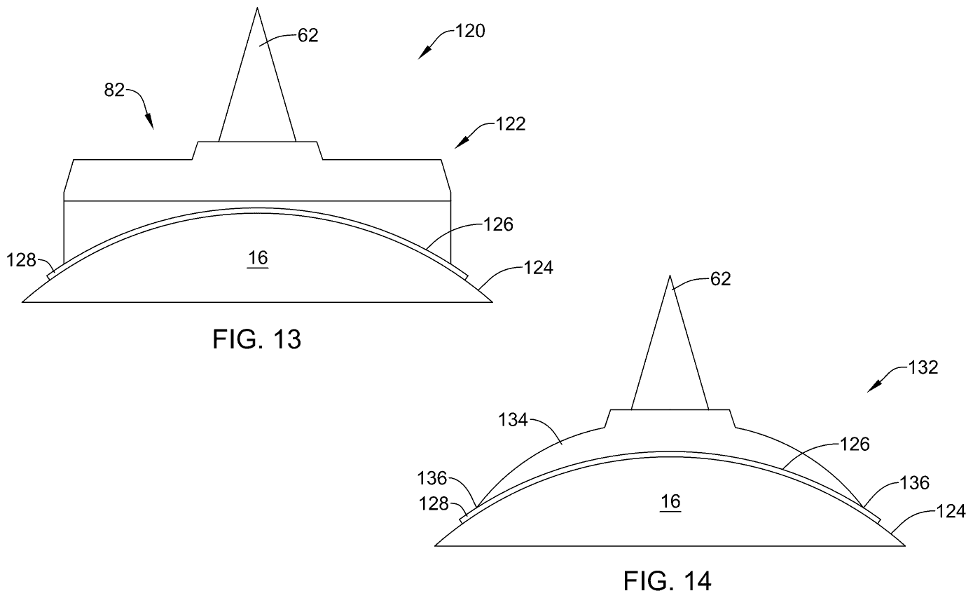

In many cases, the lower portion of the polymeric pad (such as the mounting surface 80 of the polymeric pad 64) is flat. It will be appreciated that adhesively securing a flat polymeric pad to a curved (when inflated) inflatable balloon 16 may be problematic. In some instances, the polymeric pad base may be curved to improve attachment of the polymeric pad to an inflated balloon. FIG. 13 is a schematic view of an assembly 120 that includes a blade member 62 embedded within a polymeric pad 122 that is itself secured to an outer surface 124 of the inflatable balloon 16. The polymeric pad 122 has a lower or mounting surface 126. An adhesive layer 128 extends between the outer surface 124 of the inflatable balloon 16 and the lower or mounting surface 126 of the polymeric pad 122. In some instances, the amount of adhesive between the pad and balloon may be optimized by adjusting the ratio of the pad radius of curvature and the balloon radius of curvature.

In some instances, the outer surface 124 of the inflatable balloon 16 may have, when inflated, a balloon radius of curvature that is in a range of 1 millimeter to 5 millimeters. The lower or mounting surface 126 of the polymeric pad 122 may have a pad radius of curvature that is in a range of 1 millimeter to 10 millimeters. In some instances, the balloon radius of curvature may match the pad radius of curvature to provide an even thickness of adhesive. In some instances, the balloon radius of curvature may vary somewhat from the pad radius of curvature. In some instances, the balloon radius of curvature may be slightly smaller than the pad radius of curvature. In some cases, the balloon radius of curvature may be slightly larger than the pad radius of curvature in order to provide more adhesive at the pad edges and less at the pad midline. In some instances, having a pad radius of curvature that is greater than the largest balloon radius of curvature may yield improved bonding or manufacturability between the polymeric pad 122 and a full range of inflatable balloon 16 diameters being manufactured compared to a flat pad base as well as improving folding profiles.

In FIG. 13, the polymeric pad 122 has the upper-facing surface 82 shown for example in FIG. 7. In some instances, as shown in FIG. 14, a polymeric pad 132 has an upper-facing surface 134 that curves and even tapers from a midpoint near the blade member 62 to either edge 136. While each edge 136 is shown as tapering to an effectively zero thickness, in some instances the upper-facing surface 134 may instead taper to a minimum (but non-zero) thickness, such as the edges 92 shown in FIG. 9. It will be appreciated that having the lower or mounting surface 126 of the polymeric pad 122 and 132 have a curvature that matches or nearly matches a curvature of the (inflated) inflatable balloon 16 may be utilized with the polymeric pad 64 shown in earlier drawings. Put another way, the polymeric pads 122 and 132 may include the pad flex points described with respect to the polymeric pad 64.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical device, comprising:
a catheter shaft having a distal region;
an inflatable balloon secured to the distal region; and
a blade and pad assembly extending longitudinally relative to the inflatable balloon, the blade and pad assembly comprising a polymeric pad adapted to be adhesively secured to an outer surface of the inflatable balloon and a blade member partially embedded within the polymeric pad;
the blade member comprising:
a base portion extending from a first end of the blade member to a second end of the blade member;
a blade portion extending outwardly from the base portion, the blade portion including:
a first blade flex point;
a second blade flex point;
a first blade segment extending from the first end of the blade member to the first blade flex point;
a second blade segment extending from the second end of the blade member to the second blade flex point; and
an intermediate blade segment extending between the first blade flex point and the second blade flex point; and
the polymeric pad comprising:
a first pad flex point aligned with the first blade flex point; and
a second pad flex point aligned with the second blade flex point.

2. The medical device of claim 1, wherein:
the first blade flex point comprises a first gap in the blade portion; and
the second blade flex point comprises a second gap in the blade portion, with the base portion extending without any gap.

3. The medical device of claim 1, wherein the first pad flex point comprises a first region of the polymeric pad having a reduced pad width.

4. The medical device of claim 3, wherein the first region of the polymeric pad has a width that is reduced 30 to 70 percent relative to a width of the polymeric pad outside of the first region.

5. The medical device of claim 1, wherein the second pad flex point comprises a second region of the polymeric pad having a reduced pad width.

6. The medical device of claim 5, wherein the second region of the polymeric pad has a width that is reduced 30 to 70 percent relative to a width of the polymeric pad outside of the second region.

7. The medical device of claim 1, wherein the polymeric pad tapers from a maximum thickness where the blade member is embedded in the polymeric pad to a minimum thickness along either edge of the polymeric pad parallel with the blade member.

8. The medical device of claim 1, wherein:
the outer surface of the inflatable balloon, when the inflatable balloon is inflated, has a balloon radius of curvature in a range of 1 millimeter to 4 millimeters; and
the polymeric pad has a mounting surface having a pad radius of curvature in a range of 1 millimeter to 10 millimeters.

9. The medical device of claim 8, further comprising an adhesive layer disposed between the outer surface of the inflatable balloon and the mounting surface of the polymeric pad.

10. The medical device of claim 1, further comprising one or more additional blade and pad assemblies extending longitudinally relative to the inflatable balloon, each of the one or more additional blade and pad assemblies comprising:
a blade member comprising:
a base portion extending from a first end of the blade member to a second end of the blade member;
a blade portion extending outwardly from the base portion, the blade portion including:
a first blade flex point;
a second blade flex point;
a first blade segment extending from the first end of the blade member to the first blade flex point;
a second blade segment extending from the second end of the blade member to the second blade flex point; and
an intermediate blade segment extending between the first blade flex point and the second blade flex point; and
a polymeric pad adapted to be adhesively secured to the outer surface of the inflatable balloon with the blade member partially embedded within the polymeric pad, the polymeric pad comprising:
a first pad flex point aligned with the first blade flex point; and
a second pad flex point aligned with the second blade flex point.

11. A medical device, comprising:
an inflatable balloon secured to a distal region of a catheter shaft having a longitudinal axis;
a plurality of blade and pad assemblies secured to the inflatable balloon and extending longitudinally with respect to the inflatable balloon, each of the plurality of blade and pad assemblies having a longitudinal axis and comprising:
a polymeric pad adapted to be secured to the inflatable balloon; and
two or more blade segments secured within the polymeric pad, the two or more blade segments separated by one or more blade flex points;
the polymeric pad comprising one or more pad flex points aligned with the one or more blade flex points.

12. The medical device of claim 11, wherein each of the one or more pad flex points are adapted to facilitate side bending of the corresponding polymeric pads.

13. The medical device of claim 11, wherein each of the one or more pad flex points comprise an increased-flexibility portion of the corresponding polymeric pad.

14. The medical device of claim 11, wherein each of the one or more pad flex points comprise a narrowed portion of the corresponding polymeric pad.

15. The medical device of claim 11, wherein each of the one or more pad flex points are adapted to allow adjacent polymeric pads to nest together when the inflatable balloon is deflated and folded.

16. The medical device of claim 11, wherein each of the one or more pad flex points comprise a scalloped portion of the polymeric pad.

17. A medical device, comprising:
an inflatable balloon secured to a distal region of a catheter shaft having a longitudinal axis, the inflatable balloon having an outer surface with, when inflated, a balloon radius of curvature in a range of 1 millimeter to 4 millimeters;
a polymeric pad having a mounting surface adapted to be secured to the inflatable balloon, the mounting surface having a pad radius of curvature in a range of 1 millimeter to 10 millimeters;
an adhesive layer securing the polymeric pad to the outer surface of the inflatable balloon; and
a blade member embedded within the polymeric pad, the blade member including two or more blade segments separated by one or more blade flex points;
the polymeric pad comprising one or more pad flex points aligned with the one or more blade flex points.

18. The medical device of claim 17, wherein the adhesive layer has a uniform thickness.

19. The medical device of claim 17, wherein the polymeric pad further comprises a tapered surface opposite the mounting surface.

20. The medical device of claim 17, wherein each of the one or more pad flex points comprise a region of the polymeric pad having a width that is reduced 30 to 70 percent relative to a width of the polymeric pad away from the pad flex point.
